(19) United States
(12) Patent Application Publication
(10) Pub. No.: US 2024/0076747 A1
(43) Pub. Date: Mar. 7, 2024

(54) METHODS OF DETERMINING TREATMENT OUTCOME

(71) Applicant: The Johns Hopkins University, Baltimore, MD (US)

(72) Inventors: Burles Johnson, Baltimore, MD (US); Adam Aragaki, Baltimore, MD (US); David McConkey, Baltimore, MD (US); Noah Hahn, Baltimore, MD (US); Jean Hoffman-Censits, Baltimore, MD (US)

(21) Appl. No.: 18/284,131

(22) PCT Filed: Mar. 10, 2022
(86) PCT No.: PCT/US2022/019757

§ 371 Date: Sep. 26, 2023

Related U.S. Application Data
(60) Provisional application No. 63/166,658, filed on Mar. 26, 2021.

(51) Int. Cl. C12Q 1/6886; C12Q 1/6869

(52) U.S. Cl. CPC C12Q 1/6886; C12Q 1/6869; C12Q 2600/106; C12Q 2600/158

(57) ABSTRACT

Provided herein are methods of determining treatment outcome in a subject having a disease, the method including evaluating RNA expression level of a B cell related gene to determine a B cell gene signature; evaluating RNA expression level of a CD8+ T cell related gene to determine a CD8+ T cell gene signature; analyzing the B cell gene signature and the CD8+ T cell gene signature (B8T gene signature); and determining treatment outcome in a subject based on the B8T gene signature.

### CROSS-REFERENCE TO RELATED APPLICATIONS

This application claims priority to U.S. Provisional Patent Application No. 63/166,658, filed on Mar. 26, 2021. The disclosure of this prior application is considered part of the disclosure of this application, and is incorporated in its entirety into this application.

### FEDERALLY SPONSORED RESEARCH OR DEVELOPMENT

This invention was made with government support under grant CA006973 and CA235681 awarded by the National Institutes of Health. The government has certain rights in the invention.

### TECHNICAL FIELD

The present disclosure relates to the field of biotechnology, and more specifically, to predictive biomarkers.

### BACKGROUND

Immune checkpoint inhibitor (ICI) therapies have transformed cancer care by improving overall survival (OS), particularly in patients with metastatic solid tumors. Unfortunately, only a minority of patients with metastatic solid tumors have durable responses. Several biomarkers have been developed that enrich for clinical benefit, including tumor mutational burden (TMB), intratumoral CD8+ T cell gene signatures (CD8TGS), and PD-L1. However, no biomarker, aside from mismatch repair deficiency, has been shown to predict objective response rates (ORR) in a majority of patients. Therefore, much effort is being dedicated to the development of ICI-predictive biomarkers, in order to direct ICIs to patients with the best chance of benefit while seeking alternatives for those patients who are highly unlikely to respond.

### SUMMARY

The present disclosure is based in part on the discovery that treatment outcome in a subject having a disease can be determined by analyzing both the RNA expression level of one or more B cell related genes and the RNA expression level of one or more CD8+ T cell related genes (B8T gene signature).

Without wishing to be bound by any theory, it has been discovered that analysis of the B8T gene signature can be used to determine treatment outcome in a subject having a disease.

Provided herein are methods of determining treatment outcome in a subject having a disease, the method comprising: (a) evaluating RNA expression level of a B cell related gene to determine a B cell gene signature; (b) evaluating RNA expression level of a CD8+ T cell related gene to determine a CD8+ T cell gene signature; (c) analyzing the B cell gene signature and the CD8+ T cell gene signature (B8T gene signature); and (d) determining treatment outcome in a subject based on the B8T gene signature.

Also provided herein are methods of treating a subject having a disease, the method comprising: (a) evaluating RNA expression level of a B cell related gene to determine a B cell gene signature; (b) evaluating RNA expression level of a CD8+ T cell related gene to determine a CD8+ T cell gene signature; (c) analyzing the B cell gene signature and the CD8+ T cell gene signature (B8T gene signature); (d) determining treatment outcome in a subject based on the B8T gene signature; and (e) administering a treatment to the subject when the subject is predicted to have a beneficial outcome based on the determined B8T gene signature.

In some embodiments, the disease is a cancer. In some embodiments, the cancer is a metastatic solid tumor. In some embodiments, the cancer is a bladder cancer. In some embodiments, the cancer is selected from a bladder cancer, breast cancer, cervical cancer, colon cancer, endometrial cancer, esophageal cancer, fallopian tube cancer, gall bladder cancer, gastrointestinal cancer, head and neck cancer, hematological cancer, Hodgkin lymphoma, laryngeal cancer, liver cancer, lung cancer, lymphoma, melanoma, mesothelioma, ovarian cancer, primary peritoneal cancer, salivary gland cancer, sarcoma, stomach cancer, thyroid cancer, pancreatic cancer, renal cell carcinoma, glioblastoma and prostate cancer.

In some embodiments, the treatment comprises administration of an immune checkpoint inhibitor. In some embodiments, the immune checkpoint inhibitor is atezolizumab.

In some embodiments, the B cell related gene is selected from the group consisting of BLK, CD19, FCRL2, MS4A1, KIAA0125, TNFRSF17, TCL1A, SPIB, PNOC, and combinations thereof. In some embodiments, the CD8+ T cell related gene is selected from the group consisting of CD8A, GZMA, GZMB, IFNG, CXCL9, CXCL10, PRF1, TBX21, and combinations thereof.

In some embodiments, analyzing step (c) comprises identifying the B8T gene signature to be selected from the group consisting of: B8T high/high, B8T high/low, B8T low/high, and B8T low/low. In some embodiments, the treatment outcome is determined to be beneficial to the subject when the B8T gene signature is identified as B8T high/high. In some embodiments, the beneficial treatment outcome comprises improved overall survival rate and disease control rate in the subject. In some embodiments, the treatment outcome is determined to be non-beneficial to the subject when the B8T gene signature is identified as B8T high/low or B8T low/high.

In some embodiments, analyzing step (c) further comprises identifying a prognostic biomarker in the subject, wherein the B8T gene signature and the identification of the prognostic biomarker are used in combination to determine treatment outcome in the subject. In some embodiments, the prognostic biomarker is selected from tumor mutational burden (TMB), PD-L1, or mismatch repair (MMR) deficiency. In some embodiments, analyzing step (c) further comprises identifying the gender of the subject, wherein the B8T gene signature and the gender are used in combination to determine treatment outcome in the subject. In some embodiments, the treatment outcome is determined to be beneficial to the subject when the gender of the subject is identified as male. In some embodiments, the treatment outcome is determined to be non-beneficial to the subject when the gender of the subject is identified as female. In some embodiments, the treatment outcome is determined to be beneficial to the subject when the gender of the subject is identified as female. In some embodiments, the treatment outcome is determined to be non-beneficial to the subject when the gender of the subject is identified as male.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 shows that the B8T signature stratifies patients with advanced urothelial carcinoma into cohorts with different OS in response to anti-PD-L1 therapy. FIG. 1A shows tumor B cell gene signature (BCGS, left) and CD8+ T cell gene signature (CD8TS, right) expression. FIG. 1B shows OS in response to anti-PD-L1 for patients with high versus low BCGS (left), and high versus low CD8TS (right). FIG. 1C shows a plot of each tumor by BCGS versus CD8TS. Best response in response to anti-PD-L1 therapy is in color (CR, complete response; PR, partial response; SD, stable disease; PD, progressive disease). FIG. 1D shows OS in response to anti-PD-L1 therapy stratified by B8T signature expression.

FIG. 2 shows results wherein patients with complete responses have higher CD8TGS. FIGS. 2A-2B show best response to anti-PD-L1 therapy as a function of BCGS (left) and CD8TS (right). Number of patients with each response in parentheses at top. * p<0.05, ** p<0.01. FIG. 2C shows responses to anti-PD-L1 therapy graphed as a function of B8T tumor signatures. FIGS. 2D-2E show OS in response to anti-PD-L1 therapy in patients (FIG. 2D) whose tumors grew while on cisplatin based chemotherapy before receiving ICI, and (FIG. 2E) who were not eligible for platinum based chemotherapy.

FIG. 3 shows that the B8T signature stratifies patients with TMB (high) and TMB (low) tumors into cohorts with different OS in response to anti-PD-L1 therapy. FIG. 3A shows OS in response to anti-PD-L1 therapy for patients with TMB (high) versus TMB (low) tumors. FIG. 3B shows that tumors were grouped into TMB (high) and TMB (low) groups, then graphed as a function of BCGS versus CD8TS. TMB levels are in color. FIGS. 3C-3D shows overall survival of (FIG. 3C) TMB (high) and (FIG. 3D) TMB low tumors in response to anti-PD-L1 therapy, stratified by the B8T signature. FIG. 3E shows overall survival of B8T high/high tumors in response to anti-PD-L1 treatment, stratified by the B8T signature. FIG. 3F shows overall survival of TMB (high) B8T low/low tumors versus TMB (low) B8T high/high tumors in response to anti-PD-L1 therapy.

FIG. 4 shows results wherein patients with TMB (high) tumors have higher proportion of responders. FIG. 4A shows best response grouped by TMB (low) and TMB (high) tumors. FIG. 4B shows the percentage of each B8T signature subset that comprises TMB (low) and TMB (high) tumors. FIG. 4C shows overall survival of B8T low/low tumors in response to anti-PD-L1 therapy, stratified by TMB (high) and TMB (low) tumors.

FIG. 5 shows the B8T signature stratifies patients with different PD-L1 immune cell staining into cohorts with different OS in response to anti-PD-L1 therapy. FIG. 5A shows OS in response to anti-PD-L1 therapy, separated into groups based on PD-L1 staining (IC=0, IC=1, and IC=2+). FIGS. 5B-5C show tumors were grouped into (FIG. 5B.) PD-L1 IC=2+ and (FIG. 5C) PD-L1 IC=0-1 cohorts, then OS in response to anti-PD-L1 therapy was graphed as a function of B8T signature expression. FIG. 5D shows OS of B8T high/high tumors in response to anti-PD-L1 treatment was stratified by PD-L1 IC expression (PD-L1 IC=0-1 versus PD-L1 IC=2+) FIG. 5E shows OS in response to anti-PD-L1 treatment was separated based on approved biomarkers (TMB high, PD-L1 IC=2+), the B8T signature, or a combination of the three. FIG. 5F shows a Venn diagram of the number of tumors that expressed high TMB, PD-L1 IC=2+, the B8T signature, or a combination of the three, out of 272 tumors that had evaluable information for all three biomarkers.

FIG. 6A shows best response graphed as a function of PD-L1 IC level (0, 1, or 2+).

FIG. 6B shows percentage of patients with different PD-L1 IC levels, grouped by B8T signature.

FIG. 7 shows the B8T high/high signature, or high PD-L1 immune cell levels, associates with higher OS in response to anti-PD-L1 therapy in men with metastatic urothelial cancer, but not women. FIG. 7A shows OS in response to anti-PD-L1 therapy in men and women. FIGS. 7B-7C shows OS in response to anti-PD-L1 therapy in (FIG. 7B) men and (FIG. 7C) women, stratified by B8T signature. FIG. 7D shows OS in response to anti-PD-L1 therapy was graphed based on TMB high and low tumors, separated by sex. FIGS. 7E-7F show OS in response to anti-PD-L1 therapy in (FIG. 7E) men and (FIG. 7F) women were graphed based on PD-L1 IC levels.

FIG. 8A shows tumor expression of BCGS and CD8TGS from patients in the IMvigor210 trial, separated by gender.

FIG. 8B shows OS based on high versus low BCGS, separated by men (left) and women (right).

FIG. 8C shows OS based on high versus low CD8TGS, separated by men (left) and women (right).

FIGS. 8D-8E show OS in response to anti-PD-L1 therapy in (FIG. 8D) B8T high/high tumors and (FIG. 8E) B8T low/low tumors, stratified by sex.

FIG. 8F shows best response in males (left) and females (right) were graphed as a percentage of the expressed B8T signature within each tumor.

FIGS. 9A-9C show chemotherapy naïve tumors from (FIG. 9A) all patients, (FIG. 9B) women, and (FIG. 9C) men with muscle invasive bladder cancer were separated based on B8T signature, and graphed based on overall survival.

FIG. 10A shows BCGS (top) and CD8TGS (bottom) expression of TCGA MIBC tumors.

FIGS. 10B-10C show OS for patients with MIBC, graphed based on (FIG. 10B) BCGS and (FIG. 10C) CD8TGS.

FIG. 10D show sex-specific BCGS (left) and CD8TGS (right) expression of TCGA MIBC tumors.

### DETAILED DESCRIPTION

The present disclosure is based in part on the discovery that the relationship between a B cell gene signature (e.g., RNA expression level of one or more genes in a B cell) and CD8+ T cell gene signature (e.g., RNA expression level of one or more genes in a CD8+ T cell), a B8T gene signature, can be used as a predictive biomarker for determining treatment outcome in a subject having a disease. In some embodiments, the B8T gene signature can be used in a method of treating a subject having a disease, wherein the method comprises analyzing the B8T gene signature and administering a treatment to the subject when the subject is predicted to have a beneficial outcome based on the determined B8T gene signature. While immune checkpoint inhibitor (ICI) therapies have transformed cancer care by improving overall survival (OS), much effort is being dedicated to the development of predictive biomarkers, in order to direct specific treatments to patients with the best chance of benefit while seeking alternatives for those patients who are highly unlikely to respond.

In some embodiments, provided herein are methods of determining treatment outcome in a subject having a disease that include (a) evaluating RNA expression level of a B cell related gene to determine a B cell gene signature; (b) evaluating RNA expression level of a CD8+ T cell related gene to determine a CD8+ T cell gene signature; (c) analyzing the B cell gene signature and the CD8+ T cell gene signature (B8T gene signature); and (d) determining treatment outcome in a subject based on the B8T gene signature.

Also provided herein are methods of treating a subject having a disease that include (a) evaluating RNA expression level of a B cell related gene to determine a B cell gene signature; (b) evaluating RNA expression level of a CD8+ T cell related gene to determine a CD8+ T cell gene signature; (c) analyzing the B cell gene signature and the CD8+ T cell gene signature (B8T gene signature); (d) determining treatment outcome in a subject based on the B8T gene signature; and (e) administering a treatment to the subject when the subject is predicted to have a beneficial outcome based on the determined B8T gene signature.

Various non-limiting aspects of these methods are described herein, and can be used in any combination without limitation. Additional aspects of various components of the methods described herein are known in the art.

As used herein, the term “administration” typically refers to the administration of a composition to a subject or system to achieve delivery of an agent that is, or is included in, the composition. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e. g. intrahepatic), mucosal, nasal, oral, rectal, subcutaneous, sublingual, topical, tracheal (e.g., by intratracheal instillation), vaginal, vitreal, etc. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

As used herein, the terms “cancer”, “malignancy”, “neoplasm”, “tumor”, and “carcinoma” refer to cells that exhibit relatively abnormal, uncontrolled, and/or autonomous growth, so that they exhibit an aberrant growth phenotype characterized by a significant loss of control of cell proliferation. Cancer can refer to a broad group of diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. Cancer or cancer tissue may include a tumor.

In some embodiments, a tumor may be or comprise cells that are precancerous (e.g., benign), malignant, pre-metastatic, metastatic, and/or non-metastatic. The present disclosure specifically identifies certain cancers to which its teachings may be particularly relevant. In some embodiments, a relevant cancer may be characterized by a solid tumor. In some embodiments, the cancer is a metastatic solid tumor. In some embodiments, the cancer is a bladder cancer. In some embodiments, the cancer is selected from a bladder cancer, breast cancer, cervical cancer, colon cancer, endometrial cancer, esophageal cancer, fallopian tube cancer, gall bladder cancer, gastrointestinal cancer, head and neck cancer, hematological cancer, Hodgkin lymphoma, laryngeal cancer, liver cancer, lung cancer, lymphoma, melanoma, mesothelioma, ovarian cancer, primary peritoneal cancer, salivary gland cancer, sarcoma, stomach cancer, thyroid cancer, pancreatic cancer, renal cell carcinoma, glioblastoma and prostate cancer.

As used herein, the term “chemotherapeutic agent” can refer to one or more pro-apoptotic, cytostatic and/or cytotoxic agents, for example specifically including agents utilized and/or recommended for use in treating one or more diseases, disorders or conditions associated with undesirable cell proliferation. In many embodiments, chemotherapeutic agents are useful in the treatment of cancer. In some embodiments, a chemotherapeutic agent may be or comprise one or more alkylating agents, one or more anthracyclines, one or more cytoskeletal disruptors (e.g. microtubule targeting agents such as taxanes, maytansine and analogs thereof, of), one or more epothilones, one or more histone deacetylase inhibitors HDACs), one or more topoisomerase inhibitors (e.g., inhibitors of topoisomerase I and/or topoisomerase II), one or more kinase inhibitors, one or more nucleotide analogs or nucleotide precursor analogs, one or more peptide antibiotics, one or more platinum-based agents, one or more retinoids, one or more vinca alkaloids, and/or one or more analogs of one or more of the following (i.e., that share a relevant anti-proliferative activity). In some embodiments, a chemotherapeutic agent may be utilized in the context of an antibody-drug conjugate.

As used herein, the term “determine” when used in reference to a treatment outcome refers to predicting a treatment outcome in a subject. In some embodiments, a gene signature (e.g., a B8T gene signature) is used to determine (predict) a treatment outcome in a subject.

As used herein, the term “gene signature” refers to a single or combined group of genes in a cell with a uniquely characteristic pattern of gene expression. Clinical applications of gene signatures can be characterized as prognostic, diagnostic, and predictive signatures. In some embodiments, the phenotypes that may be defined by a gene signature range from those that predict the survival or prognosis of a subject with a disease, those that are used to differentiate between different subtypes of a disease, to those that predict activation of a particular pathway. For example, gene signatures can be used to select a group of patients for whom a particular treatment will be effective.

In some embodiments, a prognostic gene signature can predict the likely outcome or course of a disease. Classifying a biological phenotype or medical condition based on a specific gene signature or multiple gene signatures, can serve as a prognostic biomarker for the associated phenotype or condition. In some embodiments, a diagnostic gene signature can serve as a biomarker that distinguishes phenotypically similar medical conditions that have a threshold of severity consisting of mild, moderate or severe phenotypes. In some embodiments, a predictive gene signature can be similar to a predictive biomarker, where it predicts the effect of treatment in patients or study participants that exhibit a particular disease phenotype. A predictive gene signature unlike a prognostic gene signature can be a target for therapy. In some embodiments, these gene signatures can have implications in facilitating personalized medicine through identification of more novel therapeutic targets and identifying the most qualified subjects for optimal benefit of specific treatments.

As used herein, the term “subject” refers to an organism, typically a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

As used herein, the term “treatment outcome” refers to an evaluation undertaken to assess the results or consequences of management and procedures used in combating disease in order to determine the efficacy, effectiveness, safety, and practicability of treatments given to a subject. In some embodiments, the determination of treatment outcome can include whether a subject will respond to the specific treatment administered to the subject. In some embodiments, determination of treatment outcome can be used to stratify patients with a disease into groups with differential treatment outcome (e.g., overall survival rate, disease control rate). In some embodiments, determination of treatment outcome can include analyzing overall survival rate, disease control rate, changes in psychological condition, or changes in physical condition (e.g., tissue damage, pain level). In some embodiments, a subject that exhibits a given gene signature (e.g., a given B8T gene signature) is predicted to have an improved outcome as compared to a reference subject that does not does not exhibit that gene signature.

### B Cell and CD8+ T Cell Gene Signatures (B8T Gene Signature)

In some embodiments, the B cell gene signature (BCGS)/CD8+ T cell gene signature (CD8TGS) or B8T gene signature is a gene signature that evaluates the RNA expression level of B cell related genes and CD8+ T cell related genes within a subject's tumor. In some embodiments, the B cell gene signature can represent the level of antigen presentation as an element of an immune response. In some embodiments, the CD8+ T cell gene signature can represent the level of adaptive anti-tumor immunity as an element of an immune response. In some embodiments, the B cell gene signature (BCGS) can be determined by evaluating RNA expression level of a B cell related gene. In some embodiments, the B cell related gene is selected from BLK, CD19, FCRL2, MS4A1, KIAA0125, TNFRSF17, TCL1A, SPIB, PNOC, and combinations thereof. Examples of B cell related genes can include, but are not limited to, BLK, BLNK, BTK, NFAM1, LYN, CDS, CD19, CD21, CD45, CD22, CD79A, CD79B, CD81, FYN, FCRL1, FCRL2, FCRL3, FCRL4, FCRL5, LAX, PIR, CR1, BAFF, CS1, LILRB1, LILRB2, LILRB3, LILRB4, LILRA3, LAIR1, and combinations thereof. In some embodiments, the CD8+ T cell gene signature (CD8TGS) can be determined by evaluating RNA expression level of a CD8+ T cell related gene. In some embodiments, the CD8+ T cell related gene is selected from CD8A, GZMA, GZMB, IFNG, CXCL9, CXCL10, PRF1, TBX21, and combinations thereof. Examples of CD8+ T cell related genes can include, but are not limited to, TNFRSF9, XCL1, XCL2, CRTAM, PSMB8, PSMB9, PSMB10, PSME2, TAP1, IRF1, FBOX6, ETV7, NKG7, GZMH, CCL4, LAG3, CD2, GBP5, CD3D, B2M, CD74, LAP3, CD7, HLA-DRA, HLA-C, HLA-DMA, and combinations thereof.

In some embodiments, the B8T gene signature can be selected from B8T high/high, B8T high/low, B8T low/high, and B8T low/low. In some embodiments, high expression of intratumoral B cell gene signature (BCGS) and high expression of CD8+ T cell gene signature (CD8TGS) in a subject can be referred to as B8T high/high. In some embodiments, high expression of intratumoral B cell gene signature (BCGS) and low expression of CD8+ T cell gene signature (CD8TGS) in a subject can be referred to as B8T high/low. In some embodiments, low expression of intratumoral B cell gene signature (BCGS) and high expression of CD8+ T cell gene signature (CD8TGS) in a subject can be referred to as B8T low/high. In some embodiments, low expression of intratumoral B cell gene signature (BCGS) and low expression of CD8+ T cell gene signature (CD8TGS) in a subject can be referred to as B8T low/low.

In some embodiments, determining the B8T gene signature of a subject can be used as a biomarker to identify a cohort of subjects with significant clinical benefit to a specific treatment (e.g., ICI therapy). For example, a B8T high/high gene signature can be a clinical diagnostic for identifying cancer patients likely to benefit from ICI therapy, either alone or in combination with other diagnostic criteria.

### Determining Treatment Outcome

Provided herein are methods of determining treatment outcome in a subject having a disease including (a) evaluating RNA expression level of a B cell related gene to determine a B cell gene signature; (b) evaluating RNA expression level of a CD8+ T cell related gene to determine a CD8+ T cell gene signature; (c) analyzing the B cell gene signature and the CD8+ T cell gene signature (B8T gene signature); and (d) determining treatment outcome in a subject based on the B8T gene signature. In some embodiments, the disease is a cancer. In some embodiments, the cancer is a metastatic solid tumor. In some embodiments, the cancer is a bladder cancer. In some embodiments, the cancer is selected from a bladder cancer, breast cancer, cervical cancer, colon cancer, endometrial cancer, esophageal cancer, fallopian tube cancer, gall bladder cancer, gastrointestinal cancer, head and neck cancer, hematological cancer, Hodgkin lymphoma, laryngeal cancer, liver cancer, lung cancer, lymphoma, melanoma, mesothelioma, ovarian cancer, primary peritoneal cancer, salivary gland cancer, sarcoma, stomach cancer, thyroid cancer, pancreatic cancer, renal cell carcinoma, glioblastoma and prostate cancer.

In some embodiments, the treatment comprises administration of an immune checkpoint inhibitor. In some embodiments, the immune checkpoint inhibitor is atezolizumab. In some embodiments, the immune checkpoint inhibitor is a PD-1 inhibitor. Examples of a PD-1 inhibitor can include, but are not limited to, pembrolizumab, nivolumab, and cemiplimab. In some embodiments, the immune checkpoint inhibitor is a PD-L1 inhibitor. Examples of a PD-L1 inhibitor can include, but are not limited to, atezolizumab, avelumab, and durvalumab. In some embodiments, the immune checkpoint inhibitor is a CTLA-4 inhibitor (e.g., ipilimumab). In some embodiments, the immune checkpoint inhibitor can be any checkpoint inhibitor, e.g., as described in Mazzarella et al., Eur J Cancer (2019) 117:14-31, hereby incorporated by reference.

In some embodiments, analyzing the B8T gene signature in a subject includes identifying the B8T gene signature to be selected from B8T high/high, B8T high/low, B8T low/high, and B8T low/low. In some embodiments, the treatment outcome is determined to be beneficial to the subject when the B8T gene signature is identified as B8T high/high. In some embodiments, the beneficial treatment outcome comprises improved overall survival rate and disease control rate in the subject. In some embodiments, the treatment outcome is determined to be non-beneficial to the subject when the B8T gene signature is identified as B8T high/low or B8T low/high. In some embodiments, the treatment outcome is determined to be non-beneficial to the subject when the B8T gene signature is identified as B8T low/low.

In some embodiments, analyzing the B8T gene signature in a subject further includes identifying a prognostic biomarker in the subject, wherein the B8T gene signature and the identification of the prognostic biomarker are used in combination to determine treatment outcome in the subject. In some embodiments, the prognostic biomarker is selected from tumor mutational burden (TMB), PD-L1, or mismatch repair (MMR) deficiency. Examples of prognostic biomarkers can include, but are not limited to, specific mutations in gene pathways (e.g., IFNGR1/2, JAK1/2, IRF1, Pbrml, Arid2, Brd7, EGFR, ALK, and ADAR1), neoantigen load, phenotype of tumor infiltrating lymphocytes (TILs), peripheral blood cell biomarkers (e.g., neutrophil count, monocyte count, and eosinophil count), and circulating tumor DNA.

In some embodiments, analyzing the B8T gene signature in a subject further includes identifying the gender of the subject, wherein the B8T gene signature and the gender are used in combination to determine treatment outcome in the subject. In some embodiments, the treatment outcome is determined to be beneficial to the subject when the gender of the subject is identified as male. In some embodiments, the treatment outcome is determined to be non-beneficial to the subject when the gender of the subject is identified as female. In some embodiments, the treatment outcome is determined to be beneficial to the subject when the gender of the subject is identified as female. In some embodiments, the treatment outcome is determined to be non-beneficial to the subject when the gender of the subject is identified as male. In some embodiments, females with B8T high/high tumors are predicted to exhibit a beneficial treatment outcome compared with the B8T high/low subtype. In some embodiments, males and females with B8T low/low tumors are predicted to exhibit a beneficial treatment outcome (e.g., OS) versus those with B8T high/low tumors. In some embodiments males with tumors with high neoantigen burden or PD-L1 IC 2+ are predicted to exhibit a beneficial treatment outcome (e.g., OS on multivariable analysis).

### Therapeutic Applications

In some embodiments, provided herein are methods of treating a subject having a disease, wherein the method includes (a) evaluating RNA expression level of a B cell related gene to determine a B cell gene signature; (b) evaluating RNA expression level of a CD8+ T cell related gene to determine a CD8+ T cell gene signature; (c) analyzing the B cell gene signature and the CD8+ T cell gene signature (B8T gene signature); (d) determining treatment outcome in a subject based on the B8T gene signature; and (e) administering a treatment to the subject when the subject is predicted to have a beneficial outcome based on the determined B8T gene signature.

Cancer can refer to a broad group of diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. Cancer or cancer tissue may include a tumor.

Cancers suitable for treatment by a method of the present disclosure can include, but are not limited to, bladder cancer, breast cancer, cervical cancer, colon cancer, endometrial cancer, esophageal cancer, fallopian tube cancer, gall bladder cancer, gastrointestinal cancer, head and neck cancer, hematological cancer, laryngeal cancer, liver cancer, lung cancer, lymphoma, melanoma, mesothelioma, ovarian cancer, primary peritoneal cancer, salivary gland cancer, sarcoma, stomach cancer, thyroid cancer, pancreatic cancer, renal cell carcinoma, glioblastoma, and prostate cancer. Non-limiting examples of cancer include: acute lymphoblastic leukemia (ALL), acute myeloid leukemia (AML), adrenocortical carcinoma, anal cancer, appendix cancer, astrocytoma, basal cell carcinoma, brain tumor, bile duct cancer, bladder cancer, bone cancer, breast cancer, bronchial tumor, Burkitt Lymphoma, carcinoma of unknown primary origin, cardiac tumor, cervical cancer, chordoma, chronic lymphocytic leukemia (CLL), chronic myelogenous leukemia (CIVIL), chronic myeloproliferative neoplasm, colon cancer, colorectal cancer, craniopharyngioma, cutaneous T-cell lymphoma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hairy cell leukemia, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, leukemia, lip and oral cavity cancer, liver cancer, lobular carcinoma in situ, lung cancer, lymphoma, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and para-nasal sinus cancer, nasopharyngeal cancer, neuroblastoma, non-Hodgkin lymphoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, and Wilms' tumor.

In some embodiments, the disease is a premalignant pathologic condition or similar pre-cancerous condition including but not limited to myelodysplastic syndromes, acquired aplastic anemia, Fanconi anemia, paroxysmal nocturnal hemoglobinuria (PNH), 5q-syndrome and any condition characterized by pathogenic cells with clonal LOH. In some embodiments, the disease is a hematologic malignancy (e.g., myelodysplastic syndrome (MDS), acute myeloid leukemia (AML), acute lymphocytic leukemia (ALL)).

In certain instances, a cancer within a subject can be monitored to evaluate the effectiveness of the cancer treatment. Any appropriate method can be used to determine whether or not a subject having cancer is treated. For example, imaging techniques or laboratory assays can be used to assess the number of cancer cells and/or the size of a tumor present within a subject. For example, imaging techniques or laboratory assays can be used to assess the location of cancer cells and/or a tumor present within a subject.

In some embodiments, a treatment can be administered to a subject having a cancer as a combination therapy with one or more additional cancer treatments. A cancer treatment can include any appropriate cancer treatments. For example, a cancer treatment can include surgery. For example, a cancer treatment can include radiation therapy. For example, a cancer treatment can include administration of one or more therapeutic agents (e.g., one or more anti-cancer agents). In some cases, an anti-cancer agent can be an immunotherapy (e.g., a checkpoint inhibitor). In some embodiments, the subject has previously been administered one or more additional anticancer therapies selected from the group consisting of ionizing radiation, a chemotherapeutic agent, a therapeutic antibody, or a checkpoint inhibitor. In some embodiments, the subject will be administered one or more additional anticancer therapies selected from the group consisting of ionizing radiation, a chemotherapeutic agent, a therapeutic antibody, or a checkpoint inhibitor.

### EXAMPLES

The disclosure is further described in the following examples, which do not limit the scope of the disclosure described in the claims.

### Example 1—High Intratumoral B8T Segregates Overall Survival Outcomes in Response to ICI

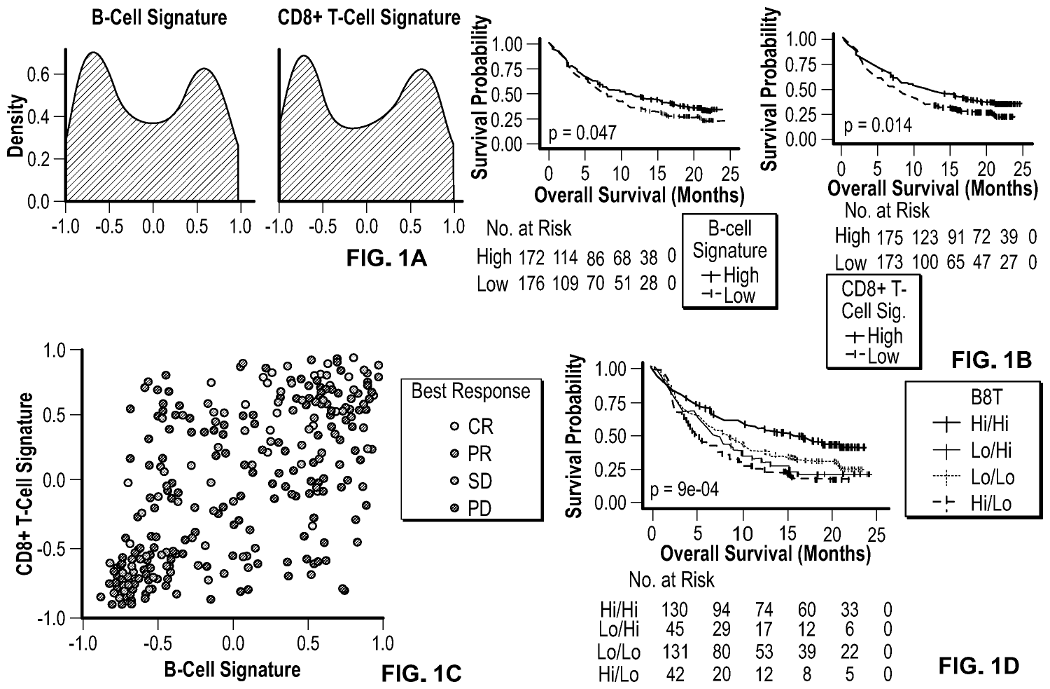

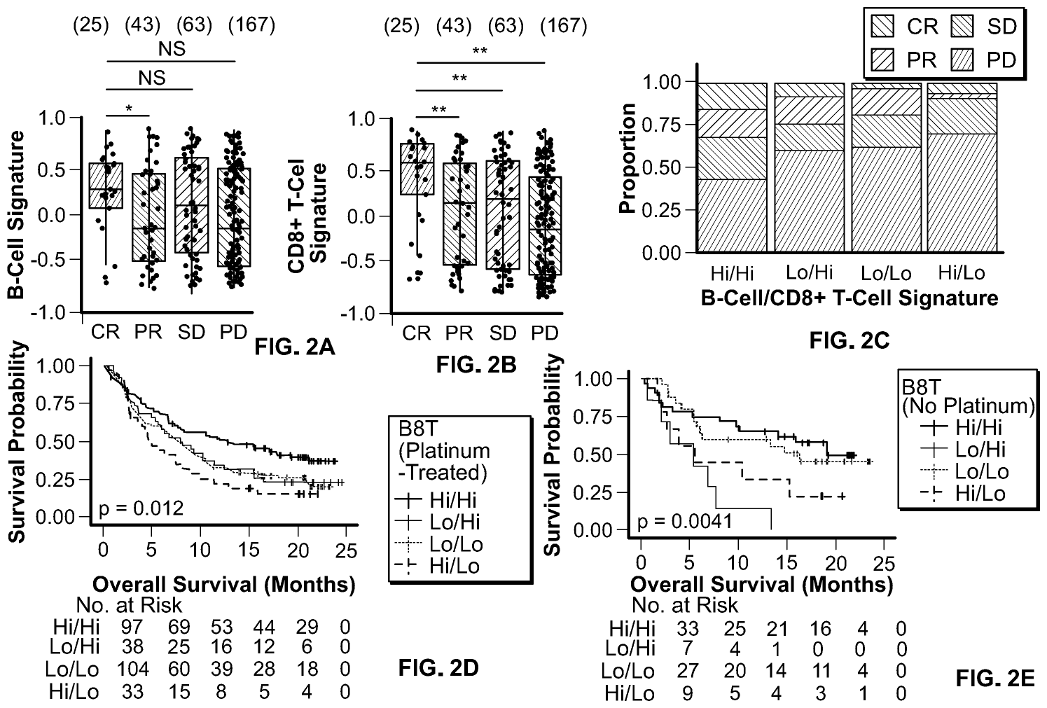

To elucidate the impact of B cells on OS in response to ICI treatment, tumor RNAseq data from the IMvigor210 trial was analyzed, where patients with advanced bladder cancer who were either cisplatin-ineligible or developed tumor progression despite cisplatin-based chemotherapy were treated with the PD-L1 inhibitor atezolizumab. Using validated BCGS and CD8TGS (Table 1), it was found that there were bimodal distributions of BCGS and CD8TGS (FIG. 1A), suggesting the existence of tumors with high and low expression of B cells and CD8+ T cells. Patients with tumors with high BCGS, or CD8TGS expression, had improved OS (FIG. 1B). This association extended to post-ICI tumor ORR outcomes, in that patients with complete responses to ICI treatment had higher BCGS or CD8TGS, versus patients with partial responses (FIG. 2A-B). However, when BCGS versus CD8TGS expression was plotted, it was found that some BCGS high tumors were CD8TGS low, and vice versa (FIG. 1C). This suggested the existence of four biologically-distinct tumor subsets, based on high or low BCGS and CD8TGS expression.

As response appeared to cluster among BCGS high CD8TGS high (B8T high/high) tumors (FIG. 1C), OS for each of the four subsets was plotted according to B8T expression. Consistent with results suggesting high intratumoral B cells and CD8+ T cells associated with improved OS in non-ICI treated melanoma, it was found that ICI-treated patients with B8T high/high bladder cancer had the longest OS and disease control rate (FIG. 1D, FIG. 2C). However, high BCGS (or CD8TGS) expression did not always confer an OS benefit, because patients with B8T high/low tumors (and B8T low/high tumors) had poor OS, even when compared with B8T low/low “immune desert” tumors (FIG. 1D). Enhanced survival with B8T high/high tumors was most striking in patients who received platinum-based chemotherapy in the metastatic setting (FIG. 2D), while chemotherapy naïve patients with B8T high/high and B8T low/low tumors who received ICI performed similarly well (FIG. 2E). Overall, these data suggest that while high co-expression of BCGS and CD8TGS correlates with improved OS in patients treated with ICI, high individual expression of either the BCGS or CD8TGS was associated with poorer outcomes.

TABLE 1

| B cell genes | CD8+ T cell genes |
|---|---|
| BLK | CD8A |
| CD19 | GZMA |
| FCRL2 | GZMB |
| MS4A1 | IFNG |
| KIAA0125 | CXCL9 |
| TNFRSF17 | CXCL10 |
| TCL1A | PRF1 |
| SPIB | TBX21 |
| PNOC | |

As response appeared to cluster among BCGS high CD8TGS high (B8T high/high) tumors (FIG. 1C), OS for each of the four subsets was plotted according to B8T expression. Consistent with results suggesting high intratumoral B cells and CD8+ T cells associated with improved OS in non-ICI treated melanoma, it was found that ICI-treated patients with B8T high/high bladder cancer had the longest OS and disease control rate (FIG. 1D, FIG. 2C). However, high BCGS (or CD8TGS) expression did not always confer an OS benefit, because patients with B8T high/low tumors (and B8T low/high tumors) had poor OS, even when compared with B8T low/low “immune desert” tumors (FIG. 1D). Enhanced survival with B8T high/high tumors was most striking in patients who received platinum-based chemotherapy in the metastatic setting (FIG. 2D), while chemotherapy naïve patients with B8T high/high and B8T low/low tumors who received ICI performed similarly well (FIG. 2E). Overall, these data suggest that while high co-expression of BCGS and CD8TGS correlates with improved OS in patients treated with ICI, high individual expression of either the BCGS or CD8TGS was associated with poorer outcomes.

### Example 2—B8T High/High Stratifies OS in Response to ICI Treatment in TMB High Tumors

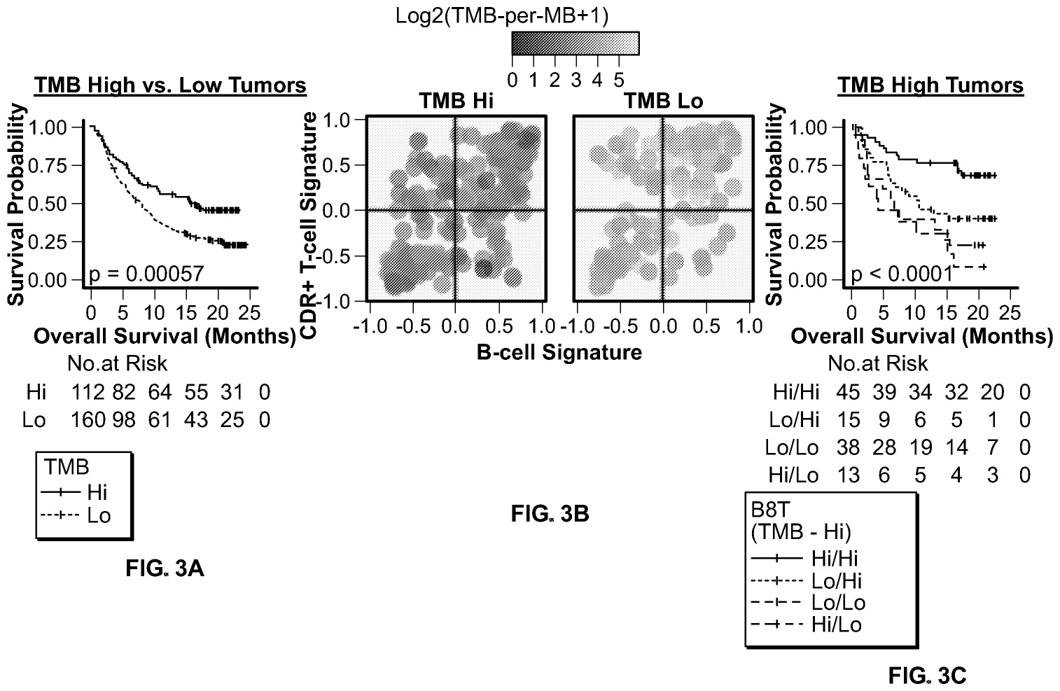

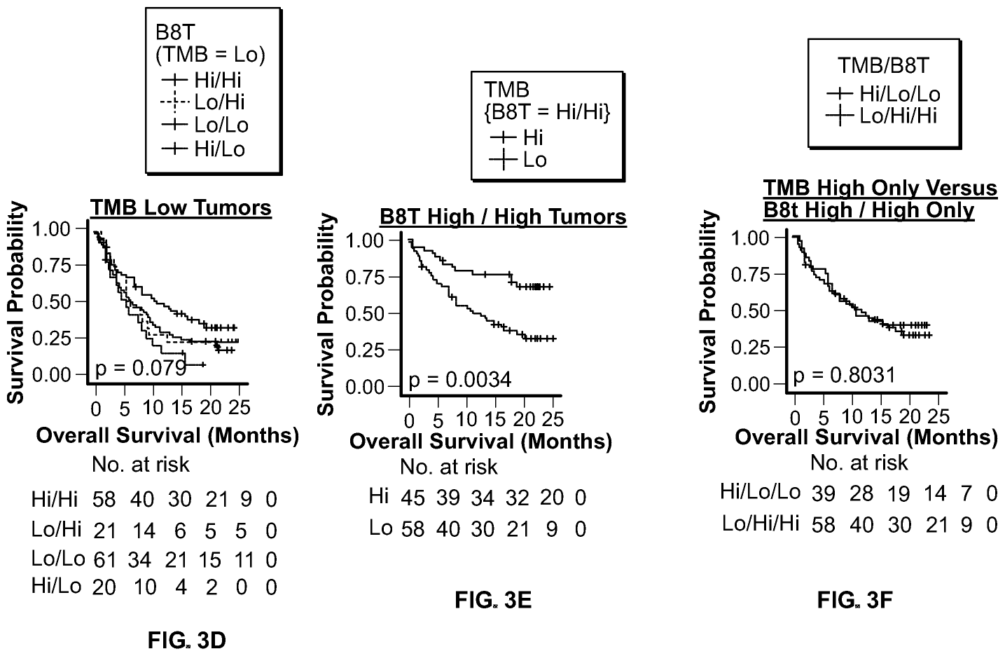

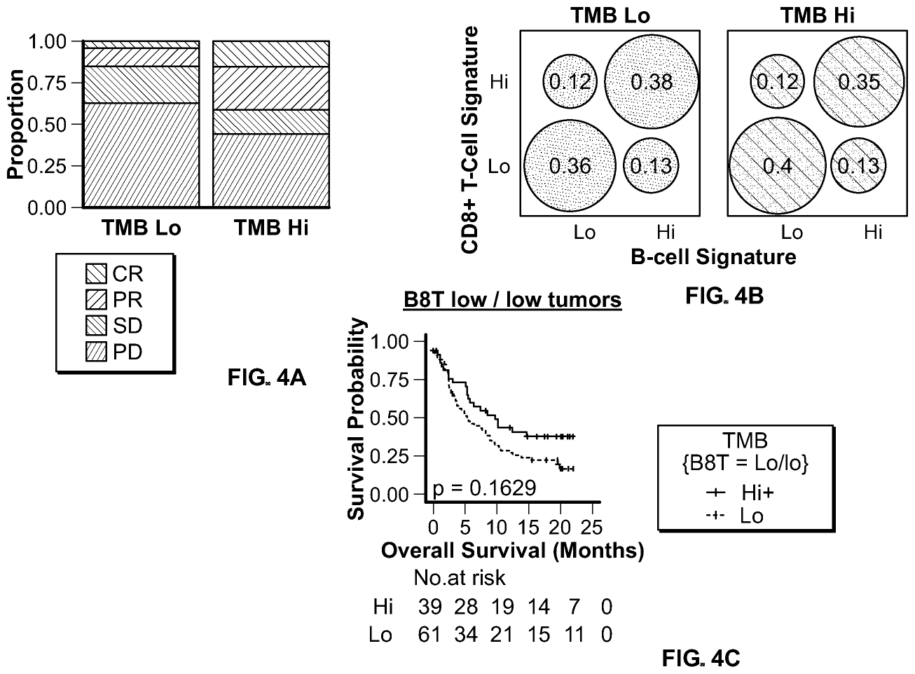

In some examples, it was confirmed that patients with high TMB tumors had higher ORR (FIG. 4A) and longer OS (FIG. 3A) than patients with low TMB in the IMvigor 210 cohort, consistent with previous observations in multiple tumor types, including patients with metastatic urothelial cancer. TMB>10 mutations per megabase was used as the cutoff for high versus low TMB as this is the FDA-approved, tumor-agnostic cutoff for use of ICI therapy in patients not otherwise eligible for ICI or another treatment. Although high TMB enriches for patients who have increased OS in response to ICI treatment, many patients with high TMB do not have increased OS (and vice versa); thus TMB alone does not adequately differentiate those with long term OS benefit. As tumors with TMB-low and TMB-high phenotypes exhibited similar heterogeneity in immune infiltration as defined by B8T expression (FIG. 3B and FIG. 4B), it was hypothesized that the B8T signature would sub-stratify tumors with TMB-high and TMB-low molecular phenotypes into subsets associated with longer and shorter OS.

To test this hypothesis, OS of TMB high patients stratified by B8T signature status was plotted and it was found the B8T high/high subset had the longest OS (FIG. 3C). The B8T high/high signature also appeared to confer a survival benefit on patients with low TMB, though this did not reach statistical significance (FIG. 3D). Conversely, TMB levels stratified B8T high/high tumors (FIG. 3E) into subsets that were also associated with different OS. TMB appeared to stratify the B8T low/low tumors into groups with different OS, but this was not statistically significant (FIG. 4C). Moreover, patients with TMB low B8T high/high tumors performed similarly to patients with TMB high B8T low/low tumors (FIG. 3F), suggesting that having either high TMB or B8T high/high conferred similar OS benefit.

### Example 3—B8T High/High Segregates OS in ICI Treated Patients in PD-L1 2+ Tumors

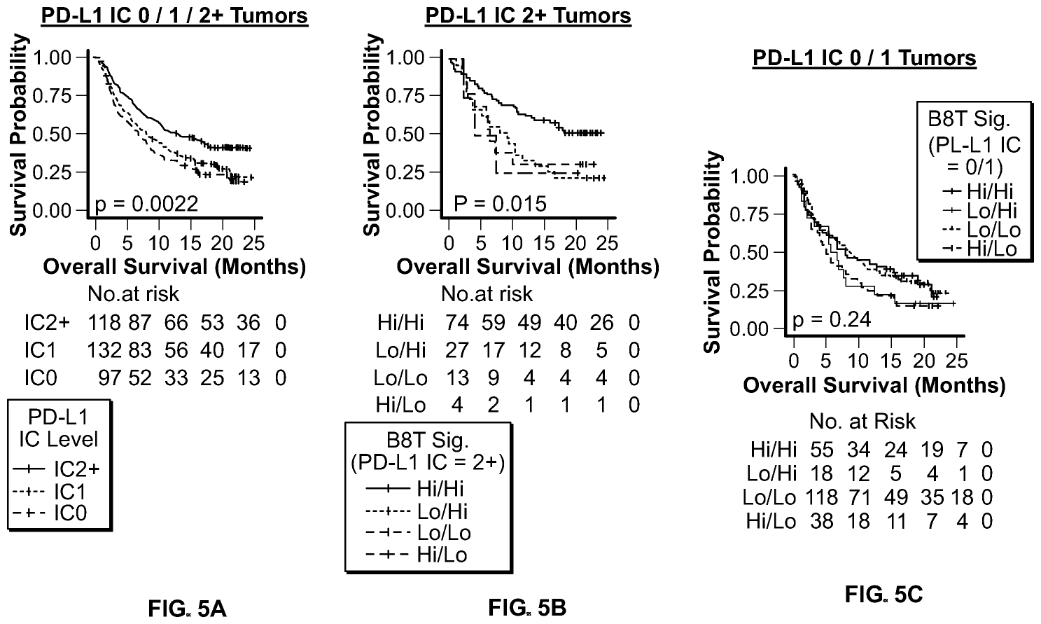

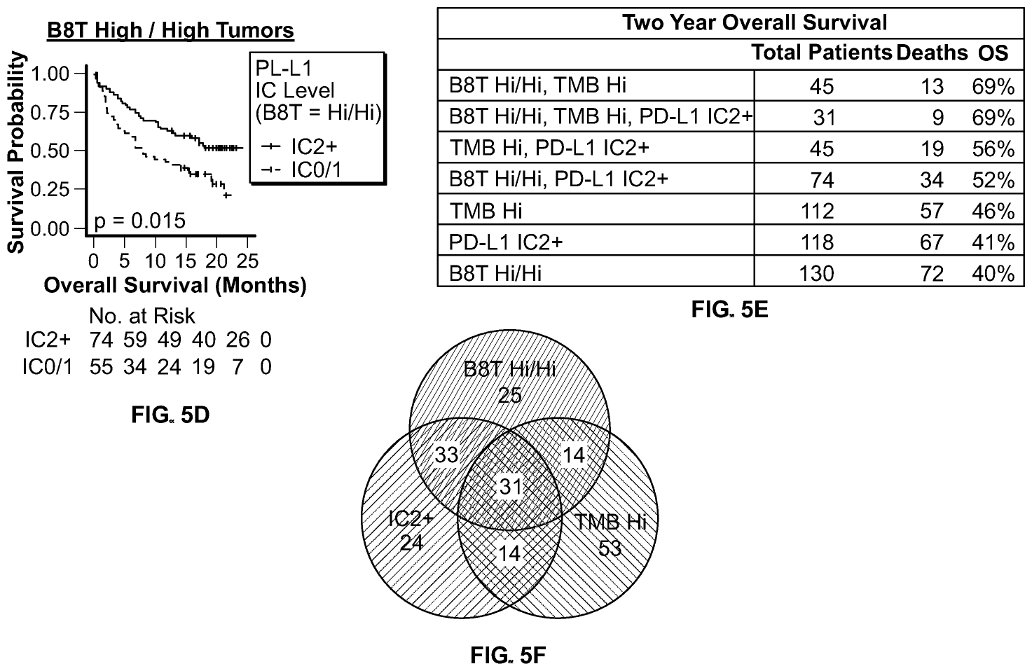

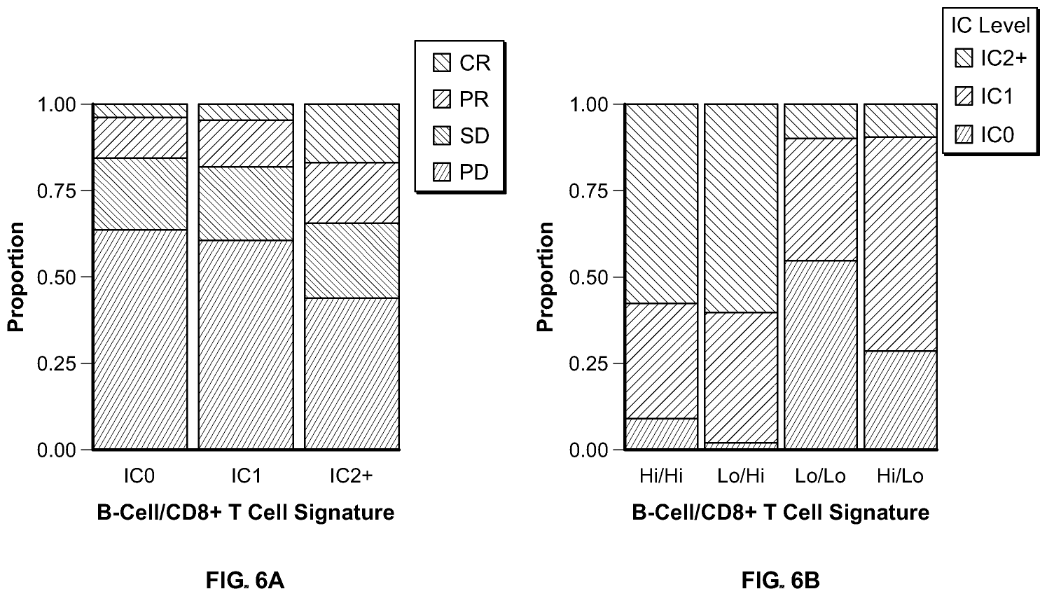

High tumor microenvironment PD-L1 is approved as a biomarker in several solid tumors as it identifies patients more likely to respond to ICI treatment. Consistent with previous reports, patients in the IMvigor210 trial whose cancers displayed high PD-L1 immune cell (IC) expression (IC 2+) experienced longer OS (FIG. 5A) and higher ORR (FIG. 6A) than patients with intermediate (IC 1) or low (IC 0) expression. Next, it was asked whether the B8T signature separated patients with PD-L1 IC high (2+) tumors into subgroups with differential OS in response to ICI therapy. It was found that while the B8T high/high tumors within the PD-L1 IC 2+ cohort as a group were associated with superior OS (FIG. 5B), the B8T high/high signature did not sub-stratify the PD-L1 IC 0/1 group (FIG. 5C). PD-L1 expression segregated outcomes within the B8T high/high tumors, because patients with B8T high/high tumors and PD-L1 IC 2+ expression had longer OS than PD-L1 IC 0-1 tumors (FIG. 5D). Of note, it was found that B8T high/high and B8T low/high tumors were enriched for PD-L1 IC 2+ staining, while B8T high/low and B8T low/low tumors were enriched for PD-L1 0-1 staining (FIG. 6B).

In order to further elucidate the significance of these biomarkers, univariable and multivariable analyses for OS (Tables 2-3) were performed. As expected, it was found that high tumor neoantigen burden and tumors expressing PD-L1 IC 2+ were associated with improved OS on multivariable analyses, while worse ECOG score correlated with worse outcomes. Notably, the B8T high/high tumors (and B8T low/low tumors) were also associated with significantly longer OS versus the B8T high/low subtype on multivariable analyses. When examining multivariable hazard ratios stratified by sex (Tables 4-5), patients (particularly females) with B8T high/high tumors had hazard ratios that trended toward benefit compared with the B8T high/low subtype, but this did not reach statistical significance. Of note, males and females with B8T low/low tumors had significantly improved OS versus those with B8T high/low tumors. Males, but not females, with tumors with high neoantigen burden or PD-L1 IC 2+ had significantly improved OS on multivariable analysis.

### Example 4—Tumors with B8T High/High Expression and High TMB are Associated with Superior OS

In order to determine the relative impact of the B8T high/high signature, high TMB, and PD-L1 IC 2+ to predict OS in patients with advanced urothelial cancer treated with atezolizumab, the effects of each biomarker individually and in combination were examined. Because these groups overlap (e.g. patients with B8T high/high+TMB high+PD-L1 IC 2+ are a subset of patients with B8T high/high+TMB high) statistical comparisons are not appropriate, so only the 2 year OS rate was provided. It was found that the B8T high/high signature, high TMB, and PD-L1 IC 2+ were each associated with similar 2 year OS (FIG. 5E). Upon combining biomarkers, it was found patients with B8T high/high+PD-L1 IC 2+ tumors, or high TMB+PD-L1 IC 2+ tumors, had similar 2 year OS that was somewhat longer than in patients captured by single biomarkers (FIG. 5E). Patients with B8T high/high+high TMB tumors appeared to have the longest OS (69% at 2 years), which was not improved upon by combining all three biomarkers (FIG. 5E).

To determine whether the B8T high/high signature pinpointed patients not identified by other biomarkers, all patients who were positive for one or more biomarker were plotted in a Venn diagram (FIG. 5F). Although there was significant overlap among groups, the results revealed the existence of a cohort of patients with B8T high/high tumors that were not positive for the other biomarkers (25 of 272 tumors evaluable for all three biomarkers, or −9%). Furthermore, −30% (14/45) of patients whose tumors co-expressed the B8T high/high signature and high TMB did not express IC 2+ levels of PD-L1, suggesting that restricting treatment to patients whose tumors expressed all three biomarkers would have excluded patients that had superior OS outcomes.

TABLE 3
Multivariable Hazard Ratios

| | |
|---|---|
| HR | CI (95%) |

B8T

| | | |
|---|---|---|
| Hi/Lo | 1 | |
| Lo/Lo | 0.5 | 0.3-0.83 |
| Lo/Hi | 0.83 | 0.41-1.67 |
| Hi/Hi | 0.45 | 0.24-0.85 |

Baseline ECOG Score

| | | |
|---|---|---|
| 0 | 1 | |
| 1 | 2.04 | 1.41-2.95 |
| 2 | 2.62 | 1.29-5.36 |

IC Level

| | | |
|---|---|---|
| IC0 | 1 | |
| IC1 | 0.88 | 0.56-1.37 |
| IC2+ | 0.5 | 0.28-0.88 |

Lund2

| | | |
|---|---|---|
| Basal/SCC-like | 1 | |
| Genomically unstable | 0.39 | 0.23-0.67 |
| Infiltrated | 0.4 | 0.23-0.68 |
| UroA | 0.43 | 0.25-0.73 |
| UroB | 1.09 | 0.44-2.7 |

Met Disease Status

| | | |
|---|---|---|
| LN Only | 1 | |
| Visceral | 1.32 | 0.79-2.19 |
| Liver | 2.29 | 1.35-3.87 |

Neoantigen Burden/MB

| | |
|---|---|
| 0.72 | 0.61-0.86 |

TABLE 2
Univariable Hazard Ratios

| | |
|---|---|
| HR | CI (95%) |

PDL1 IC Level (**) ^{1,2}

| | | |
|---|---|---|
| IC0 | 1 | |
| IC1 | 0.85 | 0.63-1.16 |
| IC2+ | 0.56 | 0.4-0.79 |

Mutation Burden/MB (***)

| | |
|---|---|
| 0.96 | 0.94-0.98 |

Baseline ECOG Score (***)

| | | |
|---|---|---|
| 0 | 1 | |
| 1 | 2.1 | 1.58-2.79 |
| 2 | 1.97 | 1.04-3.73 |

Met Disease Status (***)

| | | |
|---|---|---|
| LN Only | 1 | |
| Visceral | 1.75 | 1.16-2.63 |
| Liver | 2.89 | 1.88-4.43 |

Sample Age (#)

| | | |
|---|---|---|
| <1 yr | 1 | |
| 1-2 yr | 0.72 | 0.53-0.98 |
| >2 yr | 0.92 | 0.66-1.28 |

Received Platinum (*)

| | | |
|---|---|---|
| N | 1 | |
| Y | 1.41 | 1.01-1.98 |

Sample Collected Pre-Platinum (#)

| | | |
|---|---|---|
| N | 1 | |
| Y | 0.76 | 0.57-1.01 |

Neoantigen Burden/MB (***)

| | |
|---|---|
| 0.75 | 0.65-0.87 |

Lund2 (*)

| | | |
|---|---|---|
| Basal/SCC-like | 1 | |
| Genomically | 0.51 | 0.33-0.79 |
| Unstable | | |
| Infiltrated | 0.8 | 0.55-1.15 |
| UroA | 0.89 | 0.62-1.28 |
| UroB | 1 | 0.54-1.84 |

Lund (#)

| | | |
|---|---|---|
| MS1a | 1 | |
| MS1b | 0.95 | 0.54-1.68 |
| MS2a1 | 0.55 | 0.29-1.06 |
| MS2a2 | 0.56 | 0.26-1.19 |
| MS2b1 | 0.86 | 0.49-1.51 |
| MS2b2.1 | 1.08 | 0.51-2.27 |
| MS2b2.2 | 1.08 | 0.61-1.93 |

Immune Phenotype (#)

| | | |
|---|---|---|
| Desert | 1 | |
| Excluded | 0.92 | 0.66-1.28 |
| Inflamed | 0.65 | 0.44-0.98 |

B8T (***)

| | | |
|---|---|---|
| Hi/Lo | 1 | |
| Lo/Lo | 0.71 | 0.48-1.05 |
| Lo/Hi | 0.85 | 0.53-1.35 |
| Hi/Hi | 0.47 | 0.32-0.72 |

BCGS (*)

| | | |
|---|---|---|
| Lo | 1 | |
| Hi | 0.77 | 0.59-1 |

CD8TGS (*)

| | | |
|---|---|---|
| Lo | 1 | |
| Hi | 0.72 | 0.56-0.94 |

¹ Features not trending significant (P < 0.15) by logrank test: Sex, Race, Intravesical BCG, Tobacco Use History, TCGA Subtype, Tissue Site
² p-value by logrank test (*** < 0.001, ** < 0.01, * < 0.05, # < 0.15)

TABLE 4
Multivariable Hazard Ratios (Female)

| | |
|---|---|
| HR | CI (95%) |

B8T

| | | |
|---|---|---|
| Hi/Lo | 1 | |
| Lo/Lo | 0.16 | 0.03-0.87 |
| Lo/Hi | 0.16 | 0.01-1.89 |
| Hi/Hi | 0.09 | 0.01-1.02 |

Baseline ECOG Score

| | | |
|---|---|---|
| 0 | 1 | |
| 1 | 0.89 | 0.3-2.64 |
| 2 | 4.08 | 0.62-26.81 |

IC Level

| | | |
|---|---|---|
| IC0 | 1 | |
| IC1 | 0.54 | 0.15-1.99 |
| IC2+ | 0.61 | 0.14-2.64 |

Lund2

| | | |
|---|---|---|
| Basal/SCC-like | 1 | |
| Genomically Unstable | 0.42 | 0.09-2.02 |
| Infiltrated | 0.56 | 0.1-3.22 |
| UroA | 0.34 | 0.06-1.99 |
| UroB | 0.52 | 0.05-5 |

Met Disease Status

| | | |
|---|---|---|
| LN Only | 1 | |
| Visceral | 1.37 | 0.37-5.14 |
| Liver | 2.04 | 0.5-8.32 |

Neoantigen Burden/MB

| | |
|---|---|
| 1.09 | 0.68-1.76 |

TABLE 5
Multivariable Hazard Ratios (Male)

| | |
|---|---|
| HR | CI (95%) |

B8T

| | | |
|---|---|---|
| Hi/Lo | 1 | |
| Lo/Lo | 0.53 | 0.29-0.94 |
| Lo/Hi | 0.99 | 0.44-2.19 |
| Hi/Hi | 0.56 | 0.27-1.12 |

Baseline ECOG Score

| | | |
|---|---|---|
| 0 | 1 | |
| 1 | 2.33 | 1.51-3.29 |
| 2 | 2.7 | 1.16-6.31 |

IC Level

| | | |
|---|---|---|
| IC0 | 1 | |
| IC1 | 0.81 | 0.49-1.34 |
| IC2+ | 0.42 | 0.22-0.81 |

Lund2

| | | |
|---|---|---|
| Basal/SCC-like | 1 | |
| Genomically Unstable | 0.34 | 0.19-0.64 |
| Infiltrated | 0.39 | 0.21-0.7 |
| UroA | 0.41 | 0.22-0.76 |
| UroB | 1.35 | 0.48-3.83 |

Met Disease Status

| | | |
|---|---|---|
| LN Only | 1 | |
| Visceral | 1.31 | 0.74-2.32 |
| Liver | 2.2 | 1.2-4.01 |

Neoantigen Burden/MB

| | |
|---|---|
| 0.71 | 0.58-0.85 |

### Example 5—Gender Dependency of the Prognostic Impact of B8T High/High Expression

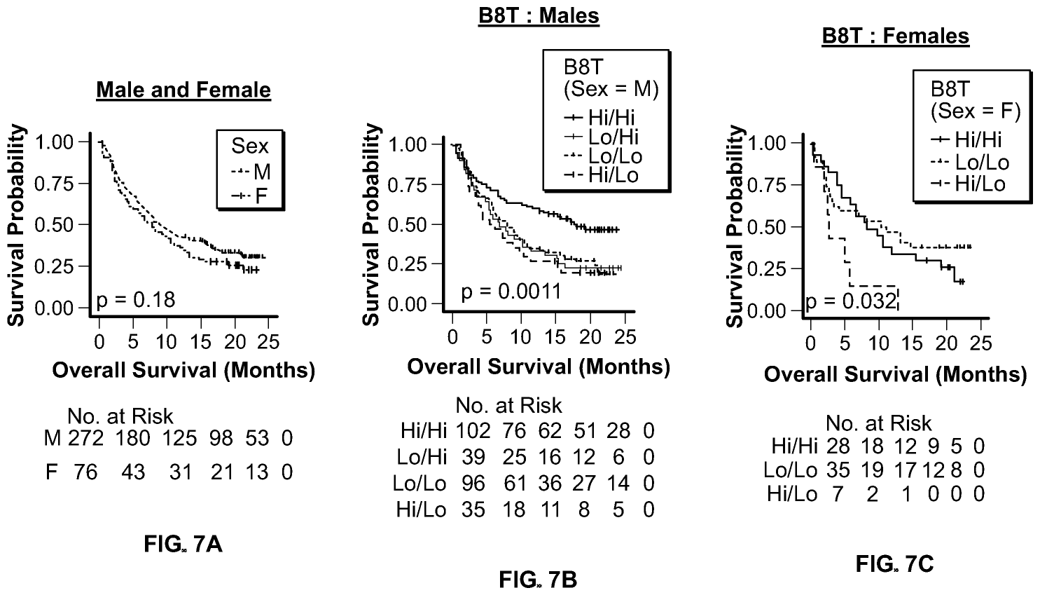

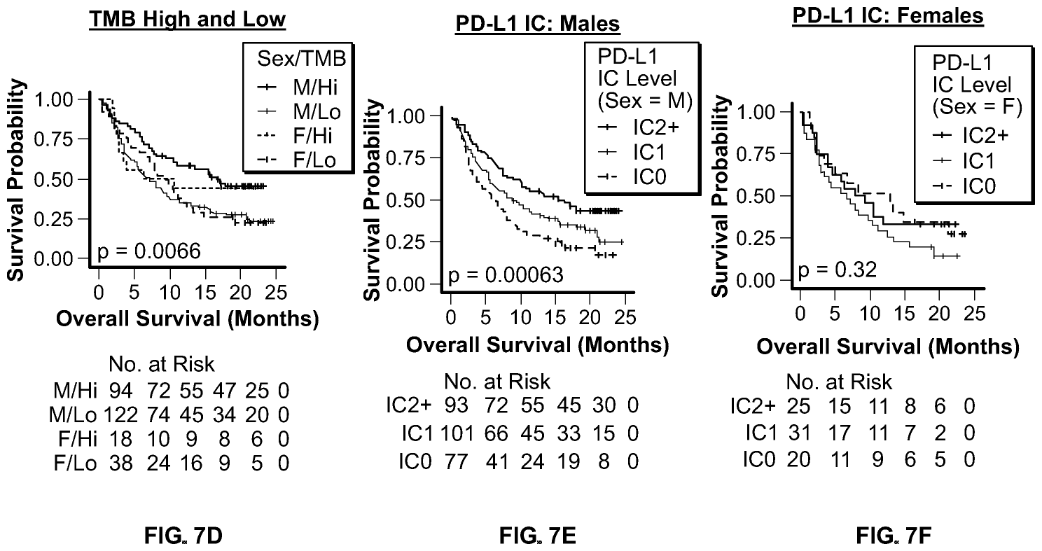

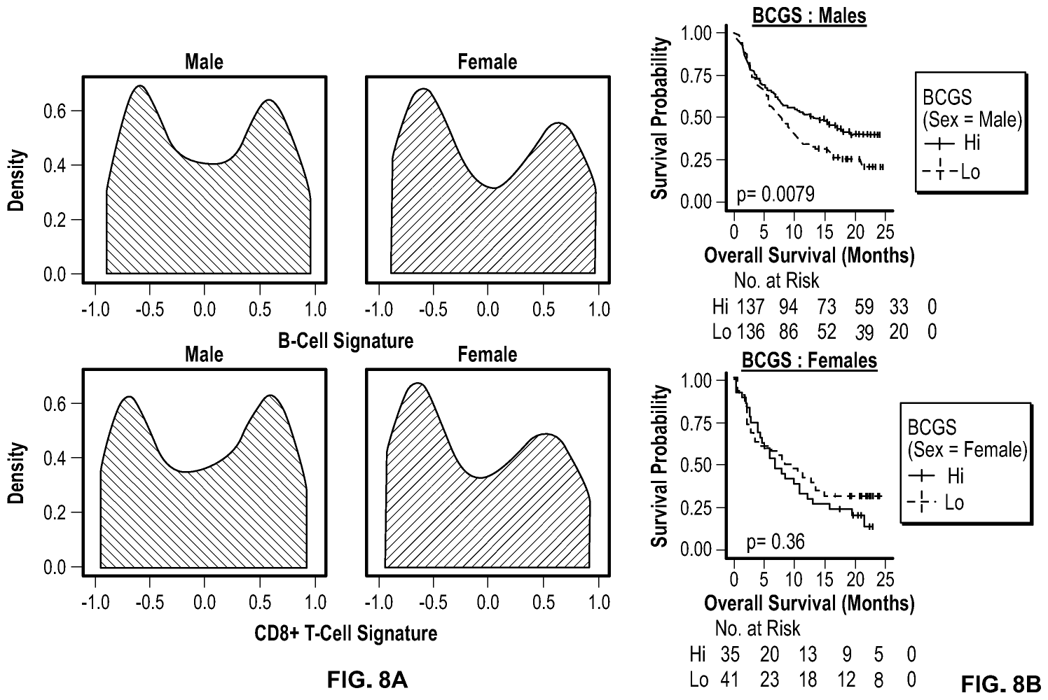

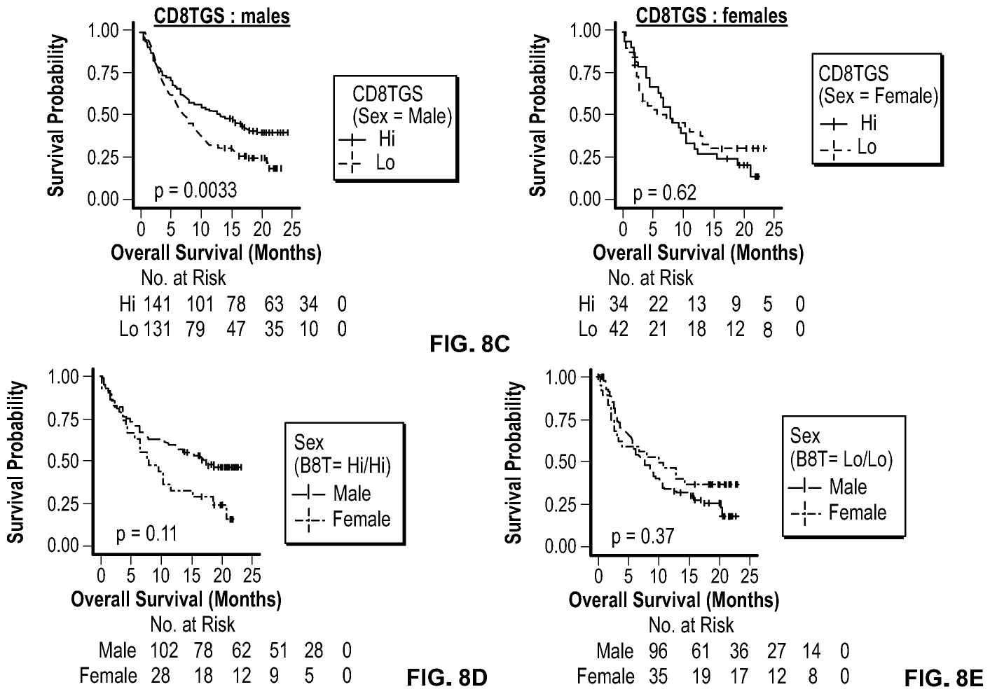

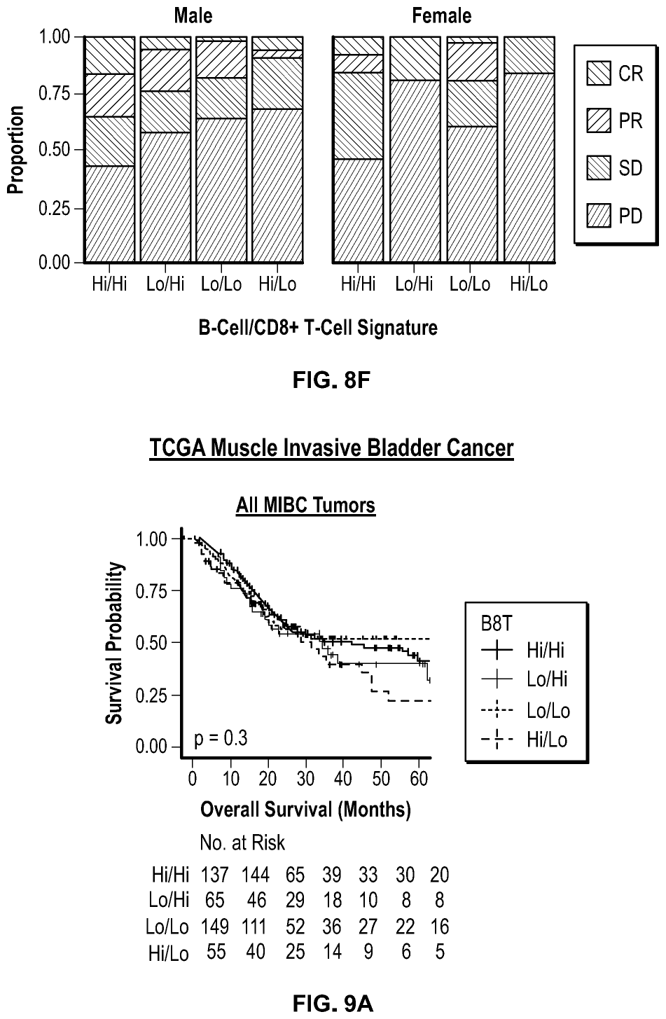

Large meta analyses have yielded inconsistent results with regard to whether or not ICI therapy produces decreased clinical benefit in women with solid tumors compared to men. Women have complex sex-specific immune-related differences when compared with men, including differential interferon alpha production by plasmacytoid dendritic cells, higher immunoglobulin levels, and different T cell-mediated cytokine production profiles, which may immunoglobulin levels, and different T cell-mediated cytokine production profiles, which may impact ICI therapeutic responses. Thus, it was examined whether the prognostic impact of the B8T signature varied according to gender. There was not a significant gender specific OS difference in the IMvigor210 cohort (FIG. 7A), and equivalent bimodal distributions in the expression of the BCGS and CD8TGS in tumors was observed from men and women (FIG. 8A). While men with B8T high/high tumors had longer OS than the other men (FIG. 7B), women with B8T high/high tumors had similar OS as compared to other women (FIG. 7C). This phenomenon was not specific to the presence of B cells or CD8+ T cells alone, because men (but not women) with high BCGS or high CD8TGS had longer OS than those with low expression of these signatures (FIG. 8B-C). Although it did not reach statistical significance, men with B8T high/high tumors, but not low/low tumors, appeared to have longer OS versus women with similar B8T expression profiles (FIG. 8D-E). Clinically, these findings correlated with the decreased ORR in women with B8T high/high tumors when compared with men (FIG. 8E).

### Example 6—the B8T Confers Sex-Specific OS Benefit in ICI Naïve Urothelial Carcinoma

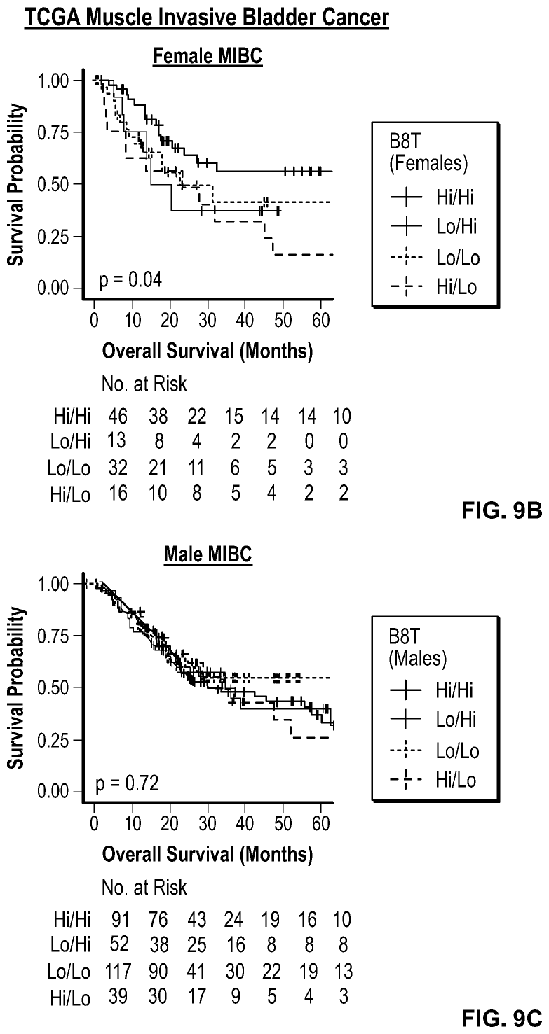

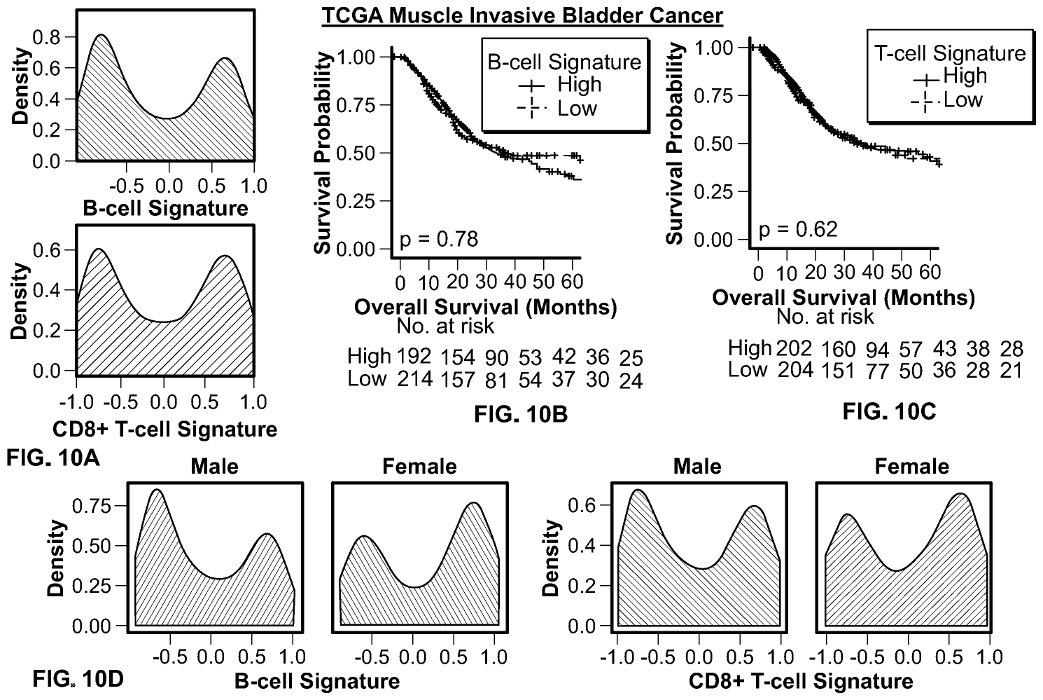

To determine if the B8T signature was associated with OS benefit in patients with urothelial cancer who did not receive ICI, the 408 tumors from the TCGA urothelial carcinoma dataset were examined, where RNA sequencing were available from chemotherapy naïve, mostly muscle-invasive bladder cancers (MIBCs) (10 were Ml, all except 1 tumor was T2-T4a). Only 12 patients received neoadjuvant chemotherapy after tissue acquisition. Similar to the IMvigor210 cohort, bimodal distributions of BCGS and CD8TGS expression was found within these tumors (FIG. 10A). Neither BCGS (FIG. 10B), CD8TGS (FIG. 10C), or the B8T signature (FIG. 9A) stratified OS in patients with MIBC. Surprisingly, when tumors were separated based on sex, it was found that the B8T stratified outcomes in women (FIG. 9B), but not men (FIG. 9C). Similar to men in the IMvigor210 cohort (FIG. 7), women with B8T high/high tumors had the best OS (FIG. 9B). Bimodal distributions of BCGS and CD8TGS were observed when tumors were divided based on sex (FIG. 10D). Finally, univariable and multivariable analyses were performed for OS benefit in patients with tumors in the TCGA cohort (Tables 6-9). On multivariable analyses, it was found that women, but not men, with B8T high/high tumors trended toward improved OS when compared with B8T high/low tumors; however this did not reach statistical significance (Tables 8-9).

TABLE 7
Multivariable Hazard Ratios

| | |
|---|---|
| HR | CI |

Age

| | |
|---|---|
| 1.03 | 1.01-1.06 |

B8T²

| | | |
|---|---|---|
| Hi/Lo | 1 | |
| Lo/Lo | 0.81 | 0.4-1.62 |
| Lo/Hi | 0.9 | 0.37-2.17 |
| Hi/Hi | 0.77 | 0.4-1.49 |

TCGA Subtype

| | | |
|---|---|---|
| Basal Squamous | 1 | |
| Luminal | 0.76 | 0.3-1.91 |
| Luminal Infiltrated | 0.99 | 0.56-1.78 |
| Luminal Papillary | 0.6 | 0.31-1.15 |
| Neuronal | 1.15 | 0.42-3.17 |

Sex

| | | |
|---|---|---|
| Female | 1 | |
| Male | 0.85 | 0.52-1.39 |

¹Days to Collection, through statistically significant (p < 0.05) had an HR that approximated 1 (0.9997)
²B8T and Sex are no significant (p < 0.15) in the univariable analysis, but are included here to be complete.

TABLE 6
Univariable Hazard Ratios

| | HR | CI (95%) |
|---|---|---|
| Sex (NS)¹ | | |
| Male | 1 | |
| Female | 1.14 | 0.83-1.58 |

Metastasis (**)

| | | |
|---|---|---|
| M0 | 1 | |
| M1 | 3.21 | 1.54-6.68 |
| Mx | 1.42 | 1.05-1.93 |

Age (***)

| | |
|---|---|
| 1.03 | 1.02-1.05 |

Grade (#)

| | | |
|---|---|---|
| Low | 1 | |
| High | 2.9 | 0.72-11.72 |

TCGA Subtype (***)

| | | |
|---|---|---|
| Basal Squamous | 1 | |
| Luminal | 1.21 | 0.69-2.12 |
| Luminal Infiltrated | 1.05 | 0.71-1.54 |
| Luminal Papillary | 0.54 | 0.37-0.81 |
| Neuronal | 1.74 | 0.98-3.09 |

B8T (NS)

| | | |
|---|---|---|
| Hi/Lo | 1 | |
| Lo/Lo | 0.7 | 0.45-1.08 |
| Lo/Hi | 0.82 | 0.5-1.36 |
| Hi/Hi | 0.68 | 0.44-1.05 |

Days to Collection (*)

| | |
|---|---|
| 1 | 1-1 |

Site of Metastasis (***)

| | | |
|---|---|---|
| None | 1 | |
| Lymph Node | 1.96 | 1.26-3.04 |
| Other | 3 | 1.42-6.33 |

Node Status (***)

| | | |
|---|---|---|
| N0 | 1 | |
| >N0 | 2.3 | 1.68-3.15 |
| Nx | 1.64 | 0.98-2.75 |

¹Features not trending significant (P < 0.15) by logrank test: Sex, Race, Age Started Smoking, B8T, Clinical Stage

TABLE 8
Multivariable Hazard Ratios (Female)

| | |
|---|---|
| HR | CI (95%) |

Age

| | |
|---|---|
| 1.02 | 0.97-1.07 |

B8T

| | | |
|---|---|---|
| Hi/Lo | 1 | |
| Lo/Lo | 1.53 | 0.37-6.35 |
| Lo/Hi | 0.14 | 0.01-1.65 |
| Hi/Hi | 0.17 | 0.02-1.18 |

TCGA Subtype

| | | |
|---|---|---|
| Basal Squamous | 1 | |
| Luminal | 0.3 | 0.03-2.82 |
| Luminal Infiltrated | 0.32 | 0.05-2.31 |
| Luminal Papillary | 0.1 | 0.01-0.73 |
| Neuronal | | |

TABLE 9
Multivariable Hazard Ratios (Male)

| | |
|---|---|
| HR | CI (95%) |

Age

| | |
|---|---|
| 1.05 | 1.02-1.08 |

B8T

| | | |
|---|---|---|
| Hi/Lo | 1 | |
| Lo/Lo | 0.66 | 0.27-1.63 |
| Lo/Hi | 1.02 | 0.37-2.83 |
| Hi/Hi | 1.01 | 0.44-2.31 |

TCGA Subtype

| | | |
|---|---|---|
| Basal Squamous | 1 | |
| Luminal | 0.43 | 0.11-1.58 |
| Luminal Infiltrated | 0.95 | 0.49-1.86 |
| Luminal Papillary | 0.93 | 0.41-2.13 |
| Neuronal | 1.5 | 0.51-4.36 |

What is claimed is:

1. A method of determining treatment outcome in a subject having a disease, the method comprising:
(a) evaluating RNA expression level of a B cell related gene to determine a B cell gene signature;
(b) evaluating RNA expression level of a CD8+ T cell related gene to determine a CD8+ T cell gene signature;
(c) analyzing the B cell gene signature and the CD8+ T cell gene signature (B8T gene signature); and
(d) determining treatment outcome in the subject based on the B8T gene signature.

2. A method of treating a subject having a disease, the method comprising:
(a) evaluating RNA expression level of a B cell related gene to determine a B cell gene signature;
(b) evaluating RNA expression level of a CD8+ T cell related gene to determine a CD8+ T cell gene signature;
(c) analyzing the B cell gene signature and the CD8+ T cell gene signature (B8T gene signature);
(d) determining treatment outcome in the subject based on the B8T gene signature; and
(e) administering a treatment to the subject when the subject is predicted to have a beneficial outcome based on the determined B8T gene signature.

3. The method of claim 1 or 2, wherein the disease is a cancer.

4. The method of claim 3, wherein the cancer is a metastatic solid tumor.

5. The method of claim 3, wherein the cancer is a bladder cancer.

6. The method of claim 3, wherein the cancer is selected from a bladder cancer, breast cancer, cervical cancer, colon cancer, endometrial cancer, esophageal cancer, fallopian tube cancer, gall bladder cancer, gastrointestinal cancer, head and neck cancer, hematological cancer, Hodgkin lymphoma, laryngeal cancer, liver cancer, lung cancer, lymphoma, melanoma, mesothelioma, ovarian cancer, primary peritoneal cancer, salivary gland cancer, sarcoma, stomach cancer, thyroid cancer, pancreatic cancer, renal cell carcinoma, glioblastoma and prostate cancer.

7. The method of any one of claims 1-6, wherein the treatment comprises administration of an immune checkpoint inhibitor.

8. The method of claim 7, wherein the immune checkpoint inhibitor is atezolizumab.

9. The method of any one of claims 1-8, wherein the B cell related gene is selected from the group consisting of BLK, CD19, FCRL2, MS4A1, KIAA0125, TNFRSF17, TCL1A, SPIB, PNOC, and combinations thereof.

10. The method of any one of claims 1-9, wherein the CD8+ T cell related gene is selected from the group consisting of CD8A, GZMA, GZMB, IFNG, CXCL9, CXCL10, PRF1, TBX21, and combinations thereof.

11. The method of any one of claims 1-10, wherein analyzing step (c) comprises identifying the B8T gene signature to be selected from the group consisting of: B8T high/high, B8T high/low, B8T low/high, and B8T low/low.

12. The method of claim 11, wherein, the treatment outcome is determined to be beneficial to the subject when the B8T gene signature is identified as B8T high/high.

13. The method of claim 12, wherein the beneficial treatment outcome comprises improved overall survival rate and disease control rate in the subject.

14. The method of claim 11, wherein the treatment outcome is determined to be non-beneficial to the subject when the B8T gene signature is identified as B8T high/low or B8T low/high.

15. The method of any one of claims 1-14, wherein analyzing step (c) further comprises identifying a prognostic biomarker in the subject, wherein the B8T gene signature and the identification of the prognostic biomarker are used in combination to determine treatment outcome in the subject.

16. The method of claim 15, wherein the prognostic biomarker is selected from tumor mutational burden (TMB), PD-L1, or mismatch repair (MMR) deficiency.

17. The method of any one of claims 1-16, wherein analyzing step (c) further comprises identifying the gender of the subject, wherein the B8T gene signature and the gender are used in combination to determine treatment outcome in the subject.

18. The method of claim 17, wherein the treatment outcome is determined to be beneficial to the subject when the gender of the subject is identified as male.

19. The method of claim 17, wherein the treatment outcome is determined to be non-beneficial to the subject when the gender of the subject is identified as female.

20. The method of claim 17, wherein the treatment outcome is determined to be beneficial to the subject when the gender of the subject is identified as female.

21. The method of claim 17, wherein the treatment outcome is determined to be non-beneficial to the subject when the gender of the subject is identified as male.
